(19) United States
(12) Patent Application Publication
(10) Pub. No.: US 2014/0342454 A1
(43) Pub. Date: Nov. 20, 2014

(54) DEVICE FOR VITRIFICATION AND/OR REANIMATION OF OOCYTES, EMBRYOS OR BLASTOCYSTS

(71) Applicant: Mariposa Biotechnology, Inc., San Clemente, CA (US)

(72) Inventors: Fred Burbank, Laguna Niguel, CA (US); Michael Jones, San Clemente, CA (US); Carl Swindle, San Clemente, CA (US)

(73) Assignee: Mariposa Biotechnology, Inc., San Clemente, CA (US)

(21) Appl. No.: 14/278,343

(22) Filed: May 15, 2014

Related U.S. Application Data
(60) Provisional application No. 61/823,822, filed on May 15, 2013.

(51) Int. Cl. C12N 5/075; C12N 5/073

(52) U.S. Cl. CPC C12N 5/0609; C12N 5/0604
USPC 435/374; 435/307.1

(57) ABSTRACT

Disclosed herein are devices, methods, systems and kits adapted for vitrification and/or reanimation of oocytes, embryos or blastocysts. The device includes a straw and a filter, wherein the straw comprises a lumen traversing through the straw and has a proximal section, a middle section and a distal section and wherein the filter is affixed in the straw and comprises a plurality of pores having a diameter smaller than the diameter of said oocytes, embryos or blastocysts but large enough to allow the passage of a fluid composition therethrough.

### CROSS-REFERENCE TO RELATED APPLICATIONS

This application claims the benefit of, and priority to, U.S. Provisional Application No. 61/823,822, filed May 15, 2013, entitled DEVICE FOR VITRIFICATION AND/OR REANIMATION OF OOCYTES, EMBRYOS OR BLASTOCYSTS, which is incorporated herein by reference in its entirety.

### TECHNICAL FIELD

The invention relates to devices adapted for vitrification and/or reanimation of oocytes, embryos or blastocysts and methods for using the device.

### BACKGROUND

In vitro fertilization (IVF) and embryo transfer are a commonly practiced treatment for a variety of causes of infertility in humans. Agricultural industries are also increasingly relying upon such assisted reproduction techniques. The ability to preserve and then reanimate oocytes, embryos or blastocysts is desirable for many reasons. Various processes for preservation and reanimation of oocytes are conventionally known. Conventionally, materials to be preserved such as oocytes, embryos or blastocysts, are subjected to cryopreservation using a slow freeze method. Typically, the materials to be cryopreserved are run through different solutions of media to dehydrate the cells of water and replace it with cryoprotectant. Then the cryoprotected materials are individually labeled and stored in cryopreservation straws, which are put in special freezers which slowly cool the embryos using liquid nitrogen. They are then stored in liquid nitrogen (−196° C.). At that extremely cold temperature, cellular activity is essentially brought to a halt, allowing them to remain viable indefinitely.

However, conventional techniques are typically labor-intensive, requiring substantial handling of the oocytes, embryos or blastocysts by a highly skilled human technician and are often difficult to reproduce in an effective, efficient and consistent manner. For example, conventional cryopreservation requires manually moving the oocytes, embryos or blastocysts from one location to another in the cryopreservation process, such as from incubation to washing solution to a cryoprotectant solution. However, such manual movement can impart osmotic and thermal shock thereby incurring structural damage to the oocytes, embryos or blastocysts. Conventional vitrification techniques are also associated with other challenges such as formation of ice crystals within the oocytes, embryos or blastocysts which can cause intracellular damage in the oocytes, embryos or blastocysts, a loss of sphericity and undesirable changes in volume. Such effects can result in structural damage in addition to toxicity, thereby significantly diminishing the viability of the oocytes, embryos or blastocysts, and ultimately reducing the probability of successful outcomes.

When the cryopreserved materials are to be used e.g. for affecting pregnancy, they are removed from the liquid nitrogen, warmed and run through solutions of media to remove the cryoprotectant and the cells are rehydrated with water. Human involvement and challenges associated with conventional preservation and reanimation techniques greatly contribute to the lack of consistency in cryopreservation and reanimation of oocytes, embryos or blastocysts and result in an undesirably low fertilization success rate. Improved systems, devices and methods for oocyte, embryo or blastocyst preservation are needed.

Vitrification is a unique process employed for cryopreserving eggs and embryos. Through vitrification, the water molecules in an embryo are removed and replaced with a higher concentration of cryoprotectant than in the slow freeze method. The embryos are then plunged directly into liquid nitrogen. This drastic freezing creates a glass transition temperature, commonly called a “glass” state, and the embryos are vitrified. This quick freezing reduces the chance for intercellular ice crystals to be formed, thus decreasing the degeneration of cells upon thawing for embryo transfer. Moreover, the survival rates of vitrified embryos are far higher than survival rates of slow freeze embryos. However, it is imperative for successful vitrification that the actually process of freezing happens very rapidly, e.g. within milliseconds. To assure that everything within the treatment vessel freezes quickly, vitrification requires rapid and uniform freezing of the material to be frozen. For vitrification to become the clinical standard for embryologists, improved systems, devices and methods are needed to ensure rapid and uniform freezing.

### SUMMARY OF THE INVENTION

There is a need for a system, device and method adapted for vitrification and/or reanimation of oocytes, embryos or blastocysts, which achieves rapid vitrification of the samples. There is also a need for a system, device and method adapted for vitrification and/or reanimation of oocytes, embryos or blastocysts, which allows for processing of multiple samples at the same time. Specifically, rather than the current process of treating and freezing one oocyte, embryo or blastocyst at a time, it would preferable to treat several, or as many as 100 samples at one time. It is also desirable to provide devices and methods for the repeatable and efficient vitrification and reanimation of oocytes, embryos or blastocysts, which mitigate effects harmful to the viability of the oocyte, embryo or blastocyst, and thereby increase the rate of successful fertilization.

This invention is directed to methods, systems and devices that address one or more of the aforementioned needs. Various methods, systems and methods described herein minimize human intervention during vitrification and/or reanimation. Moreover, the devices, systems and related methods are preferably configured to allow rapid vitrification of the oocytes, embryos or blastocysts, which minimizes damage and retains the oocytes, embryos or blastocysts with a substantial spherical shape. Additionally, some embodiments described herein allow for the simultaneous vitrification of a plurality of oocytes, embryos or blastocysts.

In one aspect, there is provided a device adapted for vitrification of oocytes, embryos or blastocysts, each having a defined diameter. The device includes a straw and a filter. The straw comprises a lumen traversing through the straw, and the straw has a proximal section, a middle section and a distal section. In some embodiments, the middle section is tapered from the proximal section to the distal section so that the proximal portion of the middle section has the same diameter as the proximal section and the distal portion of the middle section has the same diameter as the distal section. The distal section is optionally and removably capped to close the lumen running through the straw. The filter is affixed in the straw and comprises a plurality of pores having a diameter smaller than the diameter of said oocytes, embryos or blastocysts but large enough to allow the passage of a fluid composition therethrough. The straw, when capped, has an interior volume that allows the fluid composition to bathe the oocyte, embryo and/or blastocyst, and the volume ratio of the fluid composition to the oocyte, embryo and/or blastocyst is sufficient to allow vitrification of the oocyte, embryo and/or blastocyst with substantial retention of sphericity. At least a portion of the straw proximate to the filter is composed of non-insulating materials.

In another embodiment, there is provided a device adapted for vitrification of oocytes, embryos or blastocysts. The device includes a straw and a filter. The straw has a lumen traversing through the straw and comprises at least two sections: a proximal section and a distal section. In some embodiments, the distal section is tapered from the proximal section to a distal end. Alternatively, in other embodiments, the proximal section is tapered from a proximal end to the distal section. In various embodiments, the proximal section has an internal diameter of 0.05 inches to 0.07 inches and an external diameter of 0.05 inches to 0.09 inches, and the distal section has an internal diameter of 0.01 inches to 0.04 inches and an external diameter of 0.015 inches to 0.04 inches. The portion between the external diameter and the internal diameter comprises the wall of the straw. The filter is affixed in the straw and comprises a plurality of pores having a diameter smaller than a diameter of said embryos or blastocysts but large enough to allow the passage of a fluid composition.

In another aspect, this invention provides a method for vitrification of oocytes, embryos or blastocysts. The method of certain embodiments includes: (a) placing one or more of oocytes, embryos or blastocysts on a filter affixed inside a straw, and (b) continuously passing a fluid composition through the straw and over the oocytes, embryos or blastocysts.

In various embodiments, the straw comprises a lumen traversing through the straw, and the straw has a proximal section, a middle section and a distal section, which middle section is tapered from the proximal section to the distal section so that the proximal portion of the middle section has the same diameter as the proximal section and the distal portion of the middle section has the same diameter as the distal section, and which distal section is optionally and non-permanently capped to close the lumen running through the straw. In various embodiments, the filter comprises a plurality of pores having a diameter smaller than a diameter of said embryos or blastocysts but large enough to allow the passage of the fluid composition. The ratio of the volume of the fluid composition to the number of oocytes, embryos or blastocysts is adapted to obtain the desired osmolarity with the passage of a minimum amount of the fluid composition. The fluid composition is optionally modified over time in a continuous manner so that the final fluid composition corresponds to that required for vitrification of oocytes, embryos or blastocysts.

These are just some of the system's potential features and functions. The foregoing is a summary and thus contains, by necessity, simplifications, generalizations, and omissions of detail; consequently, those skilled in the art will appreciate that the summary is illustrative only and is not intended to be in any way limiting. These and the other embodiments are further described in the text that follows.

### BRIEF DESCRIPTION OF THE DRAWINGS

This invention will be further described with reference being made to the accompanying drawings.

FIG. 1A illustrates a side view of one embodiment of a vitrification device.

FIG. 1B illustrates a cross-sectional view of the vitrification device of FIG. 1A.

FIG. 1C illustrates a proximal view of the vitrification device of FIG. 1A.

FIG. 1D illustrates a distal view of the vitrification device of FIG. 1A.

FIG. 1E illustrates a magnified schematic view of the filter assembly of FIG. 1B.

FIG. 1F illustrates a perspective view of one embodiment of a vitrification system, which includes the vitrification device of FIG. 1A and one embodiment of a protective sleeve.

FIG. 2A illustrates a side view of another embodiment of a vitrification device.

FIG. 2B illustrates a cross-sectional view of the vitrification device of FIG. 2A.

FIG. 2C illustrates a magnified schematic view of the filter assembly of FIG. 2B.

FIG. 2D illustrates a perspective view of another embodiment of a vitrification system, which includes the vitrification device of FIG. 2A and an embodiment of a protective sleeve.

FIGS. 3A-C illustrate a side view, cross-sectional view, and magnified schematic view of a filter, respectively, of another embodiment of the vitrification device.

FIGS. 4A-C illustrate a side view, first cross-sectional view, and second cross-sectional view, respectively, of another embodiment of the vitrification device.

FIGS. 5A-5G illustrate schematic views of various straw embodiments, any of which may be utilized in the vitrification device, system, and method embodiments disclosed herein.

FIG. 6 illustrates a cross-sectional view of another embodiment of a vitrification device.

FIG. 7 illustrates a schematic view of a kit of parts, which comprises a vitrification device and accessories.

### DETAILED DESCRIPTION OF THE INVENTION

In the following detailed description, reference is made to the accompanying drawings, which form a part of the present disclosure. It is to be understood that the invention is not limited to the particular illustrative protocols and reagents described, as these may vary. It is also to be understood that the figures, description and terminology used herein is intended to describe particular embodiments, and are in no way intended to limit the scope of the present invention as set forth in the appended claims.

All technical and patent publications cited herein are incorporated herein by reference in their entirety. Nothing herein is to be construed as an admission that the invention is not entitled to antedate such disclosure by virtue of prior invention.

### 1. DEFINITIONS

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. It also is to be understood, although not always explicitly stated, that the reagents described herein are merely exemplary and that equivalents of such are known in the art.

In accordance with the present invention and as used herein, the following terms are defined with the following meanings, unless explicitly stated otherwise.

The term “about” includes the exact value “X” in addition to minor increments of “X” such as “X+0.1 or 1” or “X−0.1 or 1,” where appropriate. It is to be understood, although not always explicitly stated, that all numerical designations are preceded by the term “about.” All numerical designations, e.g., pH, temperature, time, concentration, and molecular weight, including ranges, are approximations which are varied (+) or (−) by increments of 0.1 or 1 where appropriate.

As used in the specification and claims, the singular form “a,” “an” and “the” include plural references unless the context clearly dictates otherwise. For example, the term “an oocyte” includes a plurality of oocytes, including populations thereof.

As used herein, the term “comprising” or “comprises” is intended to mean that the devices and methods include the recited elements but do not exclude others. “Consisting essentially of,” when used to define devices, methods, systems or kit, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than a trace amount of elements of other ingredients and substantial method steps. Embodiments defined by each of these transition terms are within the scope of this invention.

The term “population” refers to a composition of at least two individual oocytes, embryos, blastocysts or equivalents thereof. In another aspect, a “population” refers to at least three, or alternatively, at least four, or alternatively, at least five, or alternatively, at least six, or alternatively, at least seven, or alternatively, at least eight, or alternatively, at least nine, or alternatively, at least ten individual oocytes, embryos or blastocysts.

The terms “substantially spherical” and “substantial retention of sphericity” refer to an oocyte, embryo or blastocyst which has no more than ±40% change in its surface area as compared to the oocyte, embryo or blastocyst prior to introduction of a cryoprotectant during freezing or vitrifying or prior to introduction of water during thawing or reanimating. The change in the surface area of the oocyte, embryo or blastocyst can be a decrease in surface area due to shrinkage or an increase in surface area due to the introduction of undulations or other surface deformities which arise during shrinkage. In another embodiment, the oocyte, embryo or blastocyst has no more than about ±30% change, no more than about ±20% change, no more than about ±15% change, no more than about ±10% change, or no more than about ±5% change in its surface area during shrinkage.

The term “substantially non-spherical” refers to an oocyte, embryo or blastocyst which has more than ±40% change in its surface area as compared to the oocyte, embryo or blastocyst prior to introduction of a cryoprotectant during freezing or vitrifying or prior to introduction of water during thawing or reanimating. The change in the surface area of the oocyte, embryo or blastocyst can be a decrease in surface area due to shrinkage or an increase in surface area due to the introduction of undulations or other surface deformities which arise during shrinkage.

The term “partially vitrified” refers to an oocyte, embryo or blastocyst having a portion of its cytoplasmic water replaced with a cryoprotectant or pretreatment medium prior to vitrification. In some embodiments, the portion of cytoplasmic water that has been replaced with a cryoprotectant or pretreatment medium is more than about 1 v/v %, or alternatively, more than about 10 v/v %, or alternatively, more than about 50 v/v %, or alternatively, more than about 60 v/v %, or alternatively, more than about 70 v/v %, or alternatively, more than about 80 v/v %, or alternatively, more than about 90 v/v %. In one embodiment, substantially all of the cytoplasmic water of the oocyte, embryo or blastocyst (90-100 v/v %) is replaced with the cryoprotectant or pretreatment medium. In yet another embodiment, the portion of cytoplasmic water of the oocyte, embryo or blastocyst that has been replaced with cryoprotectant or pretreatment medium is sufficient to protect the oocyte, embryo or blastocyst. It is understood that one of skill in the art will be able to readily ascertain the amount of cryoprotectant or pretreatment medium necessary to protect the oocyte, embryo or blastocyst.

The term, “vitrified oocytes, embryos or blastocysts” refers to frozen oocytes, embryos or blastocysts, optionally comprising a cryoprotectant or pretreatment medium, which are preserved by rapidly cooling to low sub-zero temperatures, such as, but not limited to, 77 K or −196° C. (the boiling point of liquid nitrogen).

The term “partially reanimated” refers to an oocyte, embryo or blastocyst having at least a portion of the cytoplasmic cryoprotectant or pretreatment medium replaced with water after being thawed. In some embodiments, the portion of the cytoplasmic cryoprotectant or pretreatment medium that is replaced with water is more than about 1 v/v %, or alternatively, more than about 10 v/v %, or alternatively, more than about 50 v/v %, or alternatively, more than about 60 v/v %, or alternatively, more than about 70 v/v %, or alternatively, more than about 80 v/v %, or alternatively, more than about 90 v/v %. In one embodiment, all of the cytoplasmic cryoprotectant of the oocyte, embryo or blastocyst (100 v/v %) is replaced with water. In yet another embodiment, the cytoplasmic cryoprotectant or pretreatment medium of the “partially reanimated” oocyte, embryo or blastocyst is replaced with water sufficient to reanimate the oocyte, embryo or blastocyst. It is understood that one of skill in the art will be able to readily ascertain the amount of water necessary to reanimate the oocyte, embryo or blastocyst.

In another embodiment, the term “reanimated oocytes, embryos or blastocysts” refers to thawed oocytes, embryos or blastocysts which are capable of fertilization and/or embryo development.

As used herein, the term “vitrification” refers to rapid cooling of a liquid medium in the absence of ice crystal formation. For example, by a vitrification process, a sample containing the oocytes, blastocysts or embryos is rapidly cooled to a very low temperature such that the water content forms a glass-like state without crystallizing. Vitrification is a unique form of cryopreservation which occurs rapidly and does not require excessive handling or transfer of the oocytes, embryos or blastocysts from one solution to another.

The term “oocyte,” as used herein, refers to an unfertilized female reproductive cell and includes freshly harvested to mature oocytes. The term “freshly harvested oocyte” means that the oocyte was harvested from the animal donor within 8 hours of initiation of the stabilization/vitrification process, or alternatively within about 4 hours of initiation of the stabilization/vitrification process, or alternatively within about 1 hour of initiation of the stabilization/vitrification process, or alternatively within about 0.1 hour of initiation of the stabilization/vitrification process. The term “mature oocyte” means a harvested oocyte that is graded on a maturation scale as “mature stage—MII.” This scale further identifies harvested oocytes as “intermediate stage—(MI)” or “immature stage—(GV).” The term “egg” as used herein is meant to be synonymous with the term “oocyte.”

The term “stabilized oocytes” refers to mature oocytes still retaining their respective cumulus mass (granulosis cells), which permits maturation of the oocytes by nutrient intake through gap junctions in said cumulus masses. The mature oocyte is characterized by formation of the meiotic spindle in conjunction with extrusion of the first polar body while maintaining the integrity and activity of the intracellular proteins.

The term “blastocyst” refers to a fertilized egg during the stage of development lasting from about 5 days after fertilization up to implantation in the uterus. The term “freshly harvested blastocyst” means the blastocyst was harvested from the animal donor within about 8 hours of initiation of the stabilization/vitrification process, or alternatively, within about 4 hours of initiation of the stabilization/vitrification process, or alternatively, within about 1 hour of initiation of the stabilization/vitrification process, and alternatively, within about 0.1 hour of initiation of the stabilization/vitrification process.

The term “embryo” refers to a fertilized egg during the stage of development lasting from between the time of the first division to two cells to about 5 days after fertilization. The term “freshly harvested embryo” means the embryo was harvested from the animal donor within about 8 hours of initiation of the stabilization/vitrification process, preferably within about 4 hours of initiation of the stabilization/vitrification process, more preferably within about 1 hour of initiation of the stabilization/vitrification process, and even more preferably within about 0.1 hour of initiation of the stabilization/vitrification process.

The term “stabilization process” refers to the incubation of the oocytes, embryos or blastocysts in a stabilization solution, which provides the oocytes, embryos or blastocysts an opportunity to stabilize in a solution of low to intermediate osmolarity prior to incubation in a cryoprotecting solution having gradually increasing osmolarity.

“Osmolarity” refers to the amount of solute (dissolved chemical) per unit of total solution and is typically measured in milliosmoles per liter (mOsmol/L).

The term “cryoprotectant” or “pretreatment medium” refers to fluids or solutions used to replace extracellular and intracellular water prior to cryopreservation or vitrification. Examples of such cryoprotectants or pretreatment mediums are known in the art and include, without limitation, one or more components such as, but not limited to, sterile water, HEPES, sodium bicarbonate, sodium hydroxide, sodium chloride, potassium chloride, calcium chloride, potassium phosphate, magnesium sulfate, dextrose, sucrose, Ficoll, saline, sodium lactate solution, glycol solutions, sodium pyruvate, gentamicin sulfate, and human serum albumin. In some embodiments, the cryoprotectant or pretreatment medium is such that it does not form ice in liquid nitrogen.

The term “dehydrating agent” refers to an agent that facilitates dehydration of the intra-cytoplasmic water in the oocyte, embryo or blastocyst during cryopreservation or vitrification. In some embodiments, such agents do not osmotically traverse the cellular wall of the oocyte. Dehydrating agents include sucrose, dextrose, trehalose, lactose, raffinose, and the like.

The term “reanimating solution” refers to a solution having at least one cryoprotectant and water. A reanimating solution allows water to permeate across the cell wall of the oocyte, embryo or blastocyst, typically by osmotic methods and promotes survival and retention of viability of the oocyte, embryo or blastocyst during the process of reanimating. In some embodiments, the reanimating solution has an initial osmolarity. In another aspect, the reanimating solution comprises a dehydrating agent.

By way of example, in embodiments disclosed herein, reanimating solutions may further comprise at least one or more components such as, but not limited to, sterile water, HEPES, sodium bicarbonate, sodium hydroxide, sodium chloride, potassium chloride, calcium chloride, potassium phosphate, magnesium sulfate, dextrose, sodium lactate solution, sodium pyruvate, gentamicin sulfate and human serum albumin. Additionally or alternatively, in some embodiments, the reanimating solution does not comprise alpha globulin or beta globulin.

The term “gradually” refers to proceeding by fine or incremental steps or degrees. In some embodiments, the phrase “gradually increasing” refers to increasing the amount of a component in a solution by no more than about 0.001%, or alternatively, no more than about 0.01%, or alternatively, no more than about 0.1%, or alternatively, no more than about 1%, or alternatively, no more than about 5%, or alternatively, no more than about 10%. In some embodiments provided herein, the osmolarity of a solution is “gradually increased” at a given rate, for example, from about 90 mOsmol/L per 1 minute to about 110 mOsmol/L per 1 minute. In other embodiments, the phrase “gradually decreasing” refers to decreasing the amount of a component in a solution by no more than about 0.001%, or alternatively, no more than about 0.01%, or alternatively, no more than about 0.1%, or alternatively, no more than about 1%, or alternatively, no more than about 5%, or alternatively, no more than about 10%. In some embodiments provided herein, the osmolarity of a solution is “gradually decreased” at a given rate, for example, from about 30 mOsmol/L per 1 minute to about 50 mOsmol/L per 1 minute. Additionally or alternatively, in some embodiments provided herein, the temperature of a solution is “gradually” increased or decreased from one temperature to another temperature over a predetermined period of time. In some embodiments, the above gradual changes occur under continuous (i.e., uninterrupted) process conditions.

The term “predetermined period of time” may refer to the amount of time in which the oocytes, embryos or blastocysts are contacted with the solutions described herein in order to obtain the desired portion of a cryoprotectant, pretreatment medium or water within the oocytes, embryos or blastocysts needed to achieve a population of substantially spherical, partially vitrified or partially reanimated oocytes, embryos or blastocysts, respectively.

### 2. INTRODUCTION

Conventional cryopreservation techniques and devices have shown limited success with oocytes, embryos and blastocysts surviving after the cryopreservation and thaw process. Specifically, the large water component of oocytes, embryos and blastocysts increases the formation of intracellular ice crystals during the freezing process, which causes degeneration. Vitrifying said oocytes, embryos and/or blastocysts reduces the occurrence of intracellular ice crystals, thereby improving their viability coming out of the freeze and thaw process. In one embodiment of the vitrification process, at least some of the water molecules in an oocyte, embryo or blastocyst are removed and replaced with a higher concentration of cryoprotectant or other solution. The oocytes, embryos or blastocysts are then plunged into liquid nitrogen so that rapid freezing occurs. In other embodiments, the oocytes, embryos or blastocysts are directly plunged into liquid nitrogen without the need for replacing the water with a cryoprotectant or any other solution.

The vitrified oocytes, embryos or blastocysts are thawed and reanimated by immersion in successive warm aqueous solutions each containing, e.g., a cryoprotectant and water. The reanimation is carried out under conditions wherein osmotic shock to the oocyte(s) is inhibited. The reanimated oocytes are then stabilized in a reanimation stabilization solution maintained at a suitable temperature, e.g., from about 33° to about 38° C. for a period of time sufficient to stabilize the reanimated oocytes for fertilization.

Embodiments of the present invention provide systems and methods which are adaptable for vitrification and reanimation of oocytes, embryos and blastocysts. Embodiments of the present invention are particularly well suited for vitrification and reanimation of human oocytes, embryos and blastocysts. Embodiments provided herein may also be well suited for vitrification and reanimation of pig, cow, sheep and other mammalian oocytes, embryos and blastocysts. It will be appreciated that embodiments provided herein may be used, with or without slight modification of size, for all or nearly all vertebrates. The various exemplary embodiments will be described, and referred to, as vitrification systems. However, it should be understood that the embodiments disclosed herein are not limited to vitrification but are also adapted for reanimation. Additionally, the various embodiments may also be adapted for maturation of an egg in preparation for freezing as well as development of an embryo after fertilization and/or a blastocyst prior to implantation.

### 3. DEVICE AND SYSTEM

In one aspect, there is provided a device adapted for vitrification and/or reanimation of oocytes, embryos or blastocysts. The device includes a straw and a filter. In general, the device holds a plurality of oocytes, embryos or blastocysts. The oocytes, embryos and blastocysts each have a defined diameter.

In some embodiments, the straw is hollow. In some embodiments, the straw is formed of a straw wall, which defines a lumen traversing through the straw. The lumen includes one or more defined diameters, which permit flow of a fluid through said device. The straw has one or more sections. For example, in some embodiments, the straw has a proximal section, a middle section and a distal section. In some embodiments, the proximal section, middle section and the distal section have the same diameter. In other embodiments, the middle section is tapered from the proximal section to the distal section so that the proximal portion of the middle section has the same diameter as the proximal section and the distal portion of the middle section has the same diameter as the distal section. In some such embodiments, the proximal portion may have a uniform diameter, which is larger than a uniform diameter of the distal section. In some embodiments, the distal section is optionally occluded to close the lumen running through the said straw. In some embodiments, the open distal section of the straw can be affixed with a stopper, plug, cap or other occluding device which temporarily halts the flow of fluid out of the distal end of the straw. In some embodiments, the occluding device is fully separable from the straw; in other embodiments, the occluding device is fully detachable from the lumen but remains connected to a perimeter of the straw via a flexible connector.

The total length of the straw can be from about 0.5 inches (1.27 cm) to about 10 inches (25.4 cm) long. In some embodiments, the length of the straw is from about 2 inches to 8 inches; from about 2 inches to 6 inches; from about 2 inches to 5 inches; from about 2.5 inches to 5.5 inches; from about 3 inches to 4.5 inches; from about 3 inches to 4 inches; or from about 3 inches to 3.5 inches. In some embodiments, the length of the straw is about 1.0 to about 5 inches. The length of the proximal section can be from about 0.2 inches to about 1.0 inch. In some embodiments, the length of the proximal end to the filter can be from about 0.3 inches to about 0.9 inches; from about 0.25 inches to about 0.6 inches; from about 0.2 inches to about 0.5 inches; from about 0.3 inches to about 0.45 inches. In some embodiments, the length of the proximal section is about 0.3 to about 0.4 inches. In some embodiments, the length of the distal section is from about 0.5 inch to about 2.0 inches; from about 0.8 inches to about 1.8 inches; or from about 1.0 inch to about 1.7 inches. In some embodiments, the length of the distal section is about 1.5 inches. In some embodiments, the length of the middle section is from about 0.1 inches to about 1.0 inches; from about 0.15 inches to about 0.8 inches or from about 0.2 inches to about 0.5 inches.

It is to be understood that the optimization of the length of the straw, the length of the proximal section, the length of the distal section, or the length of the middle section may depend on the amount of the solution used for vitrification or reanimation, the amount of oocytes, embryo and blastocysts or the desired length of straw, etc. Such optimization is well within the skill of a person of ordinary skill in the art.

The proximal section and the distal section can be of a defined diameter in such a way that a diameter of the middle section is narrower than the diameter of the proximal end of the straw. In some embodiments, the proximal section and the distal section are of a defined diameter and the middle section is tapered from the proximal section to the distal section in such a way that the proximal end of the middle section has the same diameter as at least the distal end of the proximal section and the distal end of the middle section has the same diameter as at least a proximal end of the distal section. In some embodiments, one or more of the proximal section, the middle section and the distal section are tapered. In some embodiments, at least a portion of the diameter of the middle section is wider than the diameter of the distal section, but narrower than the proximal section of the device.

In some embodiments, the straw, when capped or otherwise occluded, has an interior volume that allows the fluid composition to bathe and fully cover a loaded population of oocytes, embryos and/or blastocysts. In some embodiments, the fluid composition is retained in the distal section when the distal end of the straw is capped. In other embodiments, the fluid composition is retained in the distal and the middle section when the distal end of the straw is capped. In some embodiments, the volume ratio of the fluid composition to the population of oocytes, embryos and/or blastocysts is sufficient to allow vitrification of the population with substantial retention of sphericity. In some embodiments, said volume ratio is sufficient to allow instant vitrification of the oocytes, embryos and/or blastocysts. In some embodiments, the volume ratio of the fluid composition to the oocyte, embryo and/or blastocyst is from about 1:1 to 200:1. In some embodiments, the volume ratio of the fluid composition to the population of oocytes, embryos and/or blastocysts is from about 1:2 to 1:100. In some embodiments, the volume ratio of the fluid composition to the population is from about 2:1 to 100:1. In some embodiments, the volume ratio of the fluid composition to the population is from about 10:1 to 85:1. In some embodiments, the volume ratio of the fluid composition to the population of oocytes, embryos and/or blastocysts is about 2:1, about 5:1, about 10:1, about 20:1, about 30:1, about 40:1, about 50:1, about 60:1, about 70:1, about 80:1, about 90:1 or about 100:1.

In some embodiments, at least a portion of the straw proximate to the filter is composed of non-insulating materials. In some embodiments, at least a portion of the straw proximate to the filter is composed of thermally conducting materials. The section between the external diameter and the internal diameter of the straw creates the thickness of the straw wall. In some embodiments, the thickness of the walls of the straw proximate to the filter is such that it allows easy heat exchange. Too thick a wall straw or a wall made of insulating or thermally non-conducting materials affects the freezing and thawing of the oocytes, embryos or blastocysts. In some embodiments, the wall of the straw is adapted to be thermally conductive and mechanically resistant to pressure. In one embodiment, the wall of the straw has a thickness of about 0.002 inches to about 0.02 inches. In some embodiments, the wall of the straw has a thickness of about 0.0025 inches to about 0.01 inches. In another embodiment, the wall of the straw has a thickness of about 0.003 inches to about 0.008 inches.

Suitable non-insulating or thermally conductive materials include, but are not limited to, polymers or a polymer-thermally conductive filler composite, metals, such as copper, aluminum, silver, gold, or their alloys, silicon, fiberglass, carbon nanotubes, and the like. Examples of thermally conductive fillers include metal oxides, such as aluminum oxide, magnesium oxide, zinc oxide, and quartz; metal nitrides, such as boron nitride and aluminum nitride; metal carbides, such as silicon carbide; metal hydroxides, such as aluminum hydroxide; metals, such as gold, silver, and copper; carbon fibers; and graphite.

Conventional devices and methods employ straws having thick walls and inappropriate length or volume which do not allow for rapid vitrification of the materials contained therein. Such slow freezing or vitrification often causes the liquid to crystallize which may damage the sphericity of the oocyte, embryo and/or blastocyst. Importantly, during vitrification or reanimation, maintaining the substantially spherical shape of the oocyte, embryo or blastocyst reduces or eliminates the cellular stress caused when undulations form on the surface of the oocyte, embryo or blastocyst as a result of shrinkage/compression.

In contrast to conventional devices and methods, the design and dimensions of the presently disclosed devices are such that they allow for instant, or nearly instant, vitrification of the oocyte, embryo and/or blastocyst, thereby retaining substantial sphericity. In order to achieve instant or substantially instant vitrification, it is important to achieve uniform cooling. The devices described herein achieve such uniform cooling, at least in part, through the specially-shaped design of thermally conducting materials. For example, in various embodiments, the straws are designed to maximize the surface area-to-volume ratio within the straw such that all fluid surrounding the oocytes, embryos and/or blastocysts is located substantially close (i.e., a substantially similar distance) to an inner surface of the straw wall. Similarly, the surface area-to-volume ratio within the straw is maximized such that all oocytes, embryos and/or blastocysts are located substantially close (i.e., a substantially similar distance) to an inner surface of the straw wall.

The filter is affixed inside the straw. In one embodiment, the filter is affixed in the distal section of the straw. In another embodiment, the filter is affixed in the middle section of the straw. In some embodiments, the filter affixed in the straw is replaceable, i.e., the filter can be taken out of the straw and be replaced with a new filter. In some embodiments, the filter holds a population of oocytes, embryos or blastocysts. In other embodiments, the filter holds a single oocyte, embryo or blastocyst. In some embodiments, the filter has a plurality of pores having a suitable diameter. The pores have a diameter smaller than the diameter of said oocytes, embryos or blastocysts but large enough to allow the passage of a fluid composition therethrough. Through such a design, the oocytes, embryos and/or blastocysts will settle onto a proximal face of the filter when loaded into the straw and as a fluid composition is flowed therethrough. The pores are sized to ensure that at least all healthy oocytes, embryos and blastocysts are captured, saved, and subjected to the vitrification process within the straw. In one embodiment, the pores have a diameter smaller than the diameter of said oocytes, embryos or blastocysts, but large enough to allow bathing of the oocytes, embryos and blastocysts by the fluid composition. In various embodiments, the proximal section of the straw is removable from the remainder of the straw in order to provide easy access to the oocytes, embryos or blastocysts following a reanimation process. For example, in some embodiments, the proximal section is securely but non-permanently attached to the middle or distal section via a snap fit, threaded engagement, or other removable connection. In some disposable embodiments, the proximal section is permanently removable; for example, a pull tab and/or perforated connect may facilitate breakage of the proximal section from the remainder of the straw.

As mentioned above, the filter comprises a plurality of pores wherein the pores have a diameter smaller than the diameter of the oocytes, embryos or blastocysts. This prevents the oocytes, embryos or blastocysts from passing through the filter while permitting the fluid to pass through it. In some embodiments, the plurality of pores have diameter from about 0.00001 inches to about 0.1 inches; from about 0.0001 inches to about 0.002 inches; from about 0.0001 inches to about 0.001 inches; from about 0.0005 inches to about 0.001 inches; from about 0.0007 inches to about 0.001 inches; or about 0.001 inches.

The straw can be made of any suitable materials known in the art such as glass, glass or metal coated with a thin layer of biocompatible polymers, biocompatible polymers and combinations thereof. Illustrative straw materials include, but are not limited to, polycarbonate, polyester, polyethylene terephthalate (PET), polybutylene terephthalate (PBT), polypropylene terepthalate (PPT) or polytrimethylene terepthalate (PTT), polycyclohexylenedimethylene terephthalate (PCT), poly(cyclohexylene dimethylene terephthalate)-Glycol modified polyester (PCTG), poly(ethylene terephthalate)-Glycol modified polyester (PETG), polyphenylene oxide, ethylene vinyl acetate, polypropylene and polyolefin. The filter can be made of polymeric materials including, but not limited to, polycarbonate membrane, nylon, polyolefin, stainless steel etc. In some embodiments, the filter is made of polycarbonate membrane. Preferably, the straw and the filter are made of materials that are biocompatible and non-degradable in the presence of a cryoprotecting or reanimating solution. Preferably, at least a portion of the straw is thermally conductive.

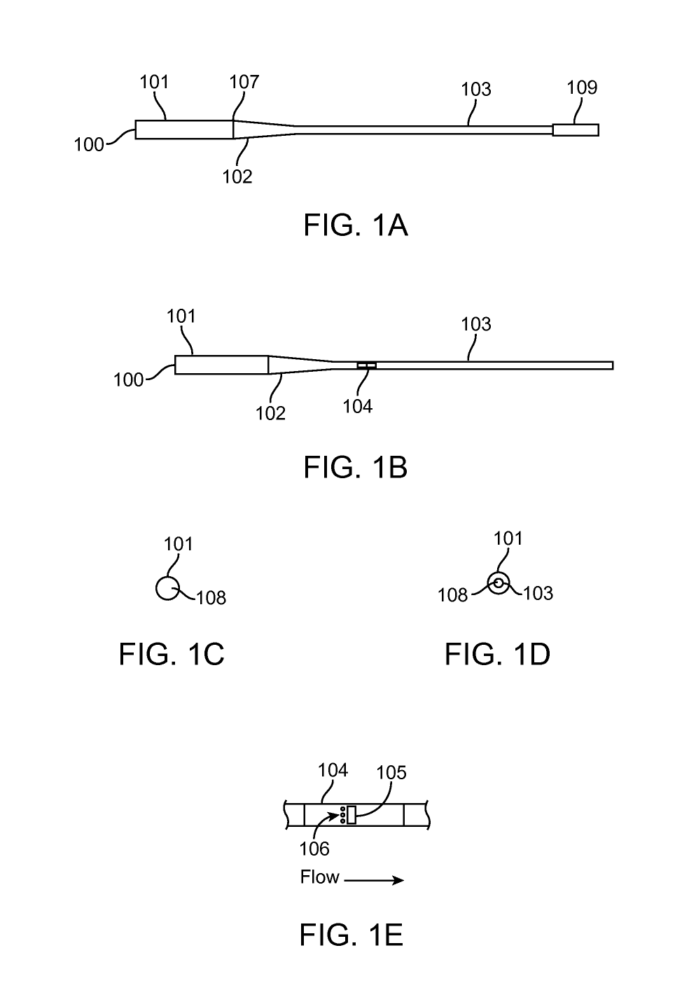

One embodiment of the device is shown schematically in the side view of FIG. 1A. As shown in the provided view, in some embodiments, the device includes a straw 100 having a proximal end and a distal end with a removable cap 109 disposed on the distal end. FIG. 1B illustrates a cross-sectional view of the device with the removable cap 109 removed. FIG. 1C illustrates a proximal view of the device with the removable cap 109 removed, and FIG. 1D illustrates a distal view with the removable cap 109 removed. The device of the depicted embodiment includes a straw 100 having an open proximal section 101, a middle section 102, and a distal section 103. The straw 100 is formed of a tubular or somewhat tubular straw wall, which defines a lumen 108 extending the length of the straw 100. The lumen 108 runs from the proximal section 101 through the middle section 102, to the distal section 103 of the straw 100. The straw further includes a filter assembly 104 affixed inside the straw 100. A magnified image of the filter assembly is shown in FIG. 1E. The filter 105 comprises a plurality of pores that have a diameter smaller than the diameter of oocytes, embryos or blastocysts 106 (for example, smaller than the diameter of human oocytes, embryos or blastocysts 106; for example, smaller than 80 microns) but large enough to allow the passage of a fluid composition therethrough. In one embodiment, the length of the proximal section 101 is about 0.8 to 0.9 inches; the length of the distal section 103 is about 1.5 inches; and the length of the middle section 102 is about 1 inch.

In some embodiments, the middle section 102 holds the plurality of the oocytes, embryos or blastocysts 106. In some embodiments, the distal section 103 of the device holds the plurality of the oocytes, embryos or blastocysts 106. In some embodiments, the proximal section 101 of the device holds the plurality of the oocytes, embryos or blastocysts. In some embodiments, the filter 105 holds the plurality of the oocytes, embryos or blastocysts. In certain embodiments, the filter 105 is disposed in the distal section 103 or the middle section 102 of the straw 100. In some embodiments, the plurality of oocytes, embryos or blastocysts 106 are mammalian. Mammals include, but are not limited to, murines, rats, simians, humans, farm animals, sport animals and pets. In some embodiments, the mammalian oocytes, embryos or blastocysts are human. The population of oocytes, embryos or blastocysts 106 can be loaded in the proximal section, for example, through the opening to the lumen 108 on the proximal end of the straw 100 (shown in FIG. 1C). In some embodiments, the oocytes, embryos or blastocysts 106 are placed on the filter 105 affixed inside the straw 100. If desired, the oocytes, embryos or blastocysts 106 can be collected after vitrification and/or reanimation by removing the filter 105 from the device, by removing the proximal portion 101 from the remainder of the straw, or by back washing the filter 105. In some embodiments, the proximal portion 101 is removable from the remainder of the straw at junction 107. As described above, in certain embodiments, the junction 107 may allow for non-permanent separation of the components; in other embodiments, the junction 107 facilitates permanent separation of the components.

One embodiment of a system 150 is provided in FIG. 1F. The system 150 includes the straw 100 described above and a protective sleeve 120. The sleeve 120 may have: an open proximal end, which receives the straw 100; an open distal end; and a lumen extending therebetween, the lumen defined by a sidewall of the protective sleeve 120. In some embodiments, a proximal portion of the straw 100 protrudes from a proximal end of the sleeve 120; in other embodiments, when the straw 100 is fully disposed within the sleeve 120, the proximal end of the straw 100 is flush with, or recessed from, the proximal end of the sleeve 120. In some embodiments, the distal end of the straw holder 120 includes a weighted feature 122, for example, a tubing portion formed of a heavy polymer, composite or metal. In some embodiments, the weighted feature 122 is formed of stainless steel. In various embodiments, the protective sleeve 120 protects the thin, fragile straw 100 as it is dropped into the liquid nitrogen for vitrification. The weighted feature 122 helps the system 150 sink into, and submerge in, the liquid nitrogen, and the opening on the distal end allows the liquid nitrogen to flow up into the lumen of the protective sleeve 120 and surround the straw 100. The sleeve 120 may also have a side opening 124 to allow for the venting of air as the system 150 descends into the liquid nitrogen. By allowing adequate air ventilation, the liquid nitrogen can move to fill the lumen of the sleeve 120 and fully surround the straw 100, or portion thereof, disposed within the sleeve 120.

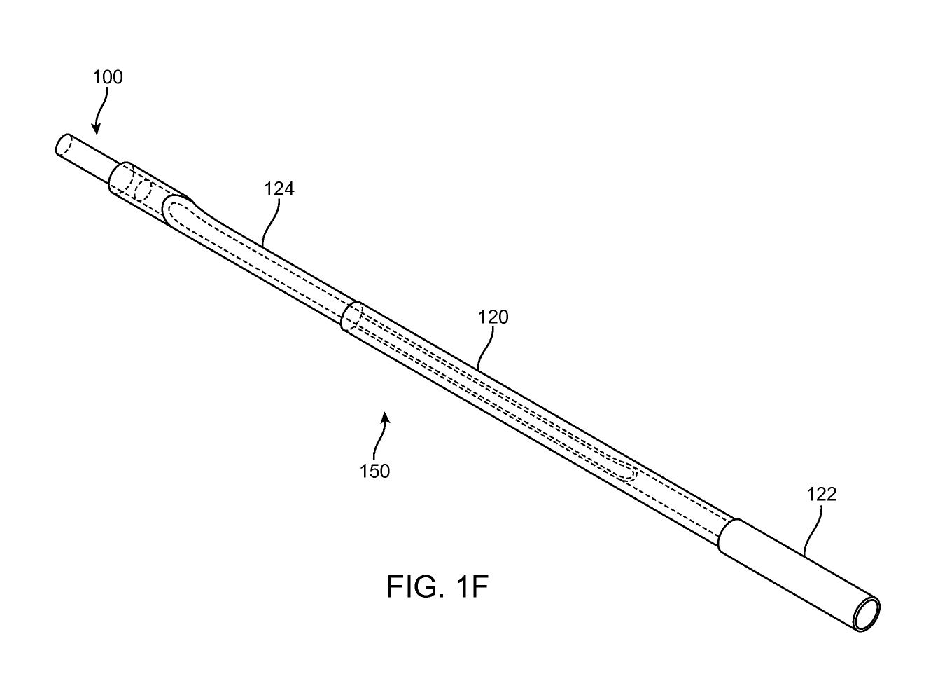

In an alternative embodiment of the vitrification device, shown in the schematic side view of FIG. 2A, the straw 200 has a lumen 208 traversing through the straw 200, the lumen 208 being defined by the straw 200. In the embodiment of FIG. 2A, the straw 200 is formed of two sections, namely, a proximal section 201 and a distal section 202. In some embodiments, the proximal section 201 is non-permanently or permanently separable from the distal section 202 at junction 207. The straw 200 of the current embodiment is tapered from the distal end of the proximal section to the distal end of the distal section. In other embodiments, the straw 200 is tapered throughout, from the proximal end of the straw 200 to the distal end. The straw further includes a filter assembly 203 affixed inside the straw 200 as seen in the cross-sectional view of FIG. 2B. A magnified image of the filter assembly is shown in FIG. 2C. The filter 204 comprises a plurality of pores that have a diameter smaller than the diameter of oocytes, embryos or blastocysts 205 loaded thereon, but large enough to allow the passage of a fluid composition therethrough.

One embodiment of a system 250 is provided in FIG. 2D. The system 250 includes the straw 200 described above and a protective sleeve 220. The sleeve 220 may have: an open proximal end, which receives the straw 200; an open distal end; and a lumen extending therebetween, the lumen defined by a sidewall of the protective sleeve 220. In some embodiments, the distal end of the protective sleeve 220 includes a weighted feature 222, for example, a tubing portion formed of stainless steel or other material of significant mass. The sleeve 220 also includes a side opening 224 for air to vent from the system 250 during submersion into liquid nitrogen for vitrification.

In various embodiments, the lengths of the proximal section and the distal section are as described hereinabove. The internal and external diameters of the proximal, middle and distal section can be suitably adjusted to achieve the desired wall thickness. In some embodiments, the proximal section has an internal diameter from about 0.03 inches to 1.0 inch, from about 0.04 inches to 0.09 inches, from about 0.05 inches to 0.07 inches or from about 0.06 inches to 0.0.065 inches and an external diameter from about 0.04 to about 1.5 inches; from about 0.05 to about 1.00 inches; or from about 0.07 inches to 0.09 inches. In some embodiments, the distal section has an internal diameter from about 0.005 to about 0.05 inches; from about 0.015 to about 0.04 inches; or from about 0.01 inches to 0.02 inches and an external diameter from about 0.01 to about 0.07 inches; from about 0.012 to about 0.05 inches; or from about 0.015 inches to 0.025 inches. In some embodiments, the portion between the external diameter and the internal diameter comprises the wall of the straw. Without being limited by any theory, the diameter of the proximal end and the distal end can be different from each other. For example, the diameter of the proximal end can be greater than the diameter of the distal end or vice versa. In the former case, the flow through the filter will be reduced by the narrower distal end thereby creating a longer residence time of the solution in contact with the oocytes.

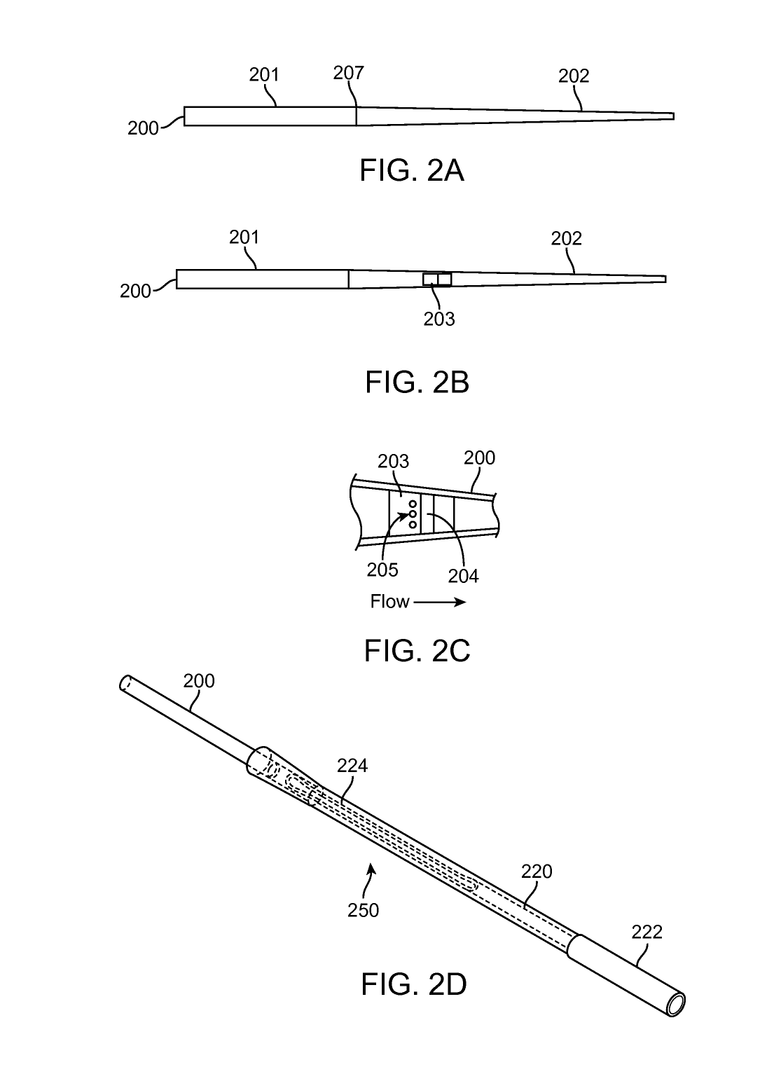

In one embodiment, the straw has a spherical-cylindrical structure. In another embodiment, the straw has a flattened-cylindrical structure. In some embodiments, the device includes a flattened cylinder as a vessel for treating material to be vitrified while minimizing the physical distance from the freezing or thawing solutions. In an alternative embodiment of the device, as shown in FIG. 3A, the straw 300 has a round proximal entrance 301 and distal exit 302 section integrated into a larger, flattened middle section 303. The round entrance and exit sections may be as small as 0.008 inches in diameter, or as small as 0.050 inches in diameter depending on the material to be processed. In some embodiments, some of or all the flattened section has a width equal or approximately equal to the diameter of the entrance and/or exit portions. The flattened middle section 303, at its largest dimension, may be, as one non-limiting example, 0.25 inches in height and 0.25 inches long in length, where length is measured from a proximal end of the middle section 303 to a distal end of the middle section 303. The straw further comprises a filter 304 affixed inside the straw. The placement of the filter 304 within the middle section 303 is shown in FIG. 3A. FIG. 3B shows a cross-section of the structure of FIG. 3A. A magnified image of the filter 304 is shown in FIG. 3C. The filter 304 comprises a plurality of pores 305 that have a diameter smaller than the diameter of oocytes, embryos or blastocysts but large enough to allow fluids to pass through.

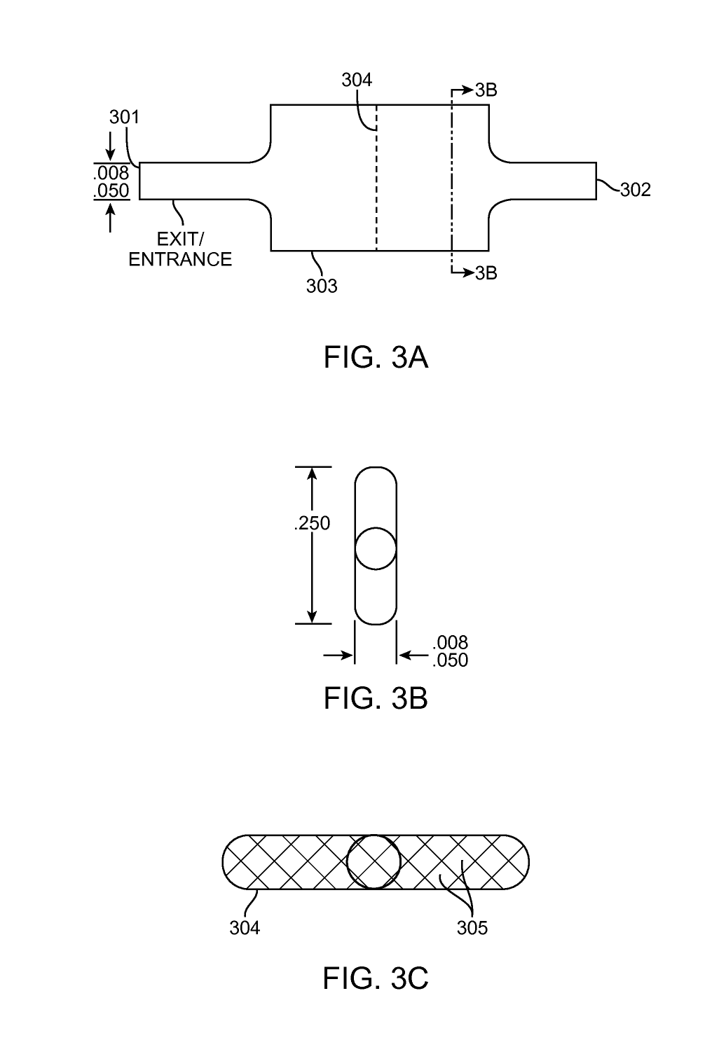

Another embodiment of the device is shown in FIGS. 4A-4C, wherein the entire straw 400 has a flattened profile approximately equal to the diameter of the edges. The dimensions provided within the figures are intended to provide a non-limiting example of acceptable dimensions. For example, as an illustration only, the straw 400 may be as small as 0.008 inches tall or as large as 0.050 inches tall, depending on the material being treated. Similarly, the straw 400 may range from 0.008 inches wide to 0.25 inches wide. As in other embodiments described herein, the total length L of the straw may range from about 0.5 inches (1.27 cm) to about 10 inches (25.4 cm), or any range of sub-values therebetween. In some embodiments (not shown), the entrance diameter can be larger or smaller than the exit diameter. In some embodiments, the filter 404 may be disposed in the middle of the straw's length; in other embodiments, the filter 404 may be disposed closer to the proximal end or closer to the distal end. As in other embodiments, the filter 404 is porous and includes a plurality of pores 405 sized to prevent the flow of oocytes, embryos and/or blastocysts from a proximal side of the filter to a distal side of the filter 404.

As shown in the several depicted embodiments above, in various embodiments, the filter extends across an entire cross-sectional area of the straw. In some embodiments, such as shown in FIG. 1E and FIG. 2C, the filter is housed within a filter assembly disposed within the straw. In some such embodiments, the filter assembly is removable from the straw, for example, to replace the filter and/or to collect the oocytes, embryos and/or blastocysts.

The straw includes a filter that allows for the passage of vitrification solutions while retaining the material within the straw. The requirements for the filter will vary depending on the material being treated. Specifically, the size of the pores or opening that allow the vitrification solutions to pass must not allow the material being treated to pass. In addition, the pores must be adequately sized so that they hold the material without damaging the material. The physical location of the filter may be anywhere along the length of the straw. Depending on the material being treated, the filter can be placed towards the end of the flattened section. In some cases the filter can be placed in the middle of the flattened section. It some embodiments, the filter can be placed in the smaller entrance/exit sections.

In some embodiments, the distance that any material within the straw is from the outer wall is such that it allows for a significant increase in space volume while providing for an even greater increase in surface area; such a design allows for efficient heat transfer during the vitrification process. Another embodiment would provide smaller proximal and distal (i.e. entrance and exit) sections while maintaining the middle flattened section.

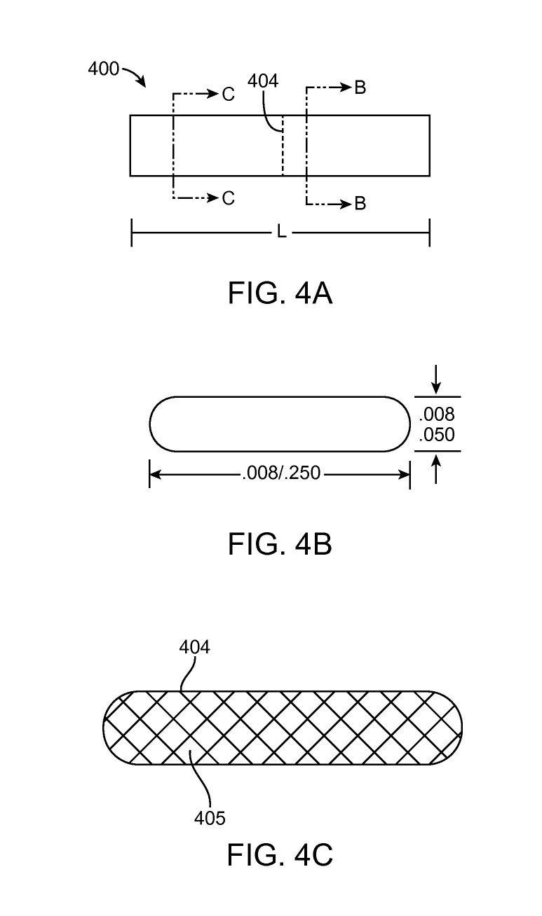

One skilled in the art will appreciate that a wide range of straw designs may be utilized and are herein expressly contemplated and incorporated into this disclosure. Any design with appropriate dimensions to achieve desired flow, to house a population of oocytes, embryos and/or blastocysts on a filter, and to achieve a satisfactorily high surface area-to-volume ratio may be used according to the principles of the present invention. In order to achieve rapid cooling without the formation of ice crystals, the device must enable efficient and substantially uniform heat transfer. Thus, various embodiments provided herein, include straws of unique shape and design, which increase the surface area-to-volume ratio, as compared to conventional vitrification straws. By increasing said ratio, the distance between the thermally-conducting straw wall and the fluid or population of oocytes, embryos or blastocysts is minimized. Thus, all fluid and oocytes, embryos or blastocysts are within a desired distance range from the wall of the straw. In various embodiments, the desired distance range is the range in which substantially uniform cooling can be achieved. Various non-limiting illustrative examples of straw designs covered by this invention are provided in FIGS. 5A-5G. As shown in FIG. 5G, in some straw embodiments, including any embodiments described elsewhere herein, the surface of the straw may be textured, for example, to include dimples, ridges, crimping, or the like, in order to further increase the surface area of the straw.

In some embodiments, the straw can be open on both ends. In various embodiments, a cap, plug, valve, or other occlusion mechanism may be provided to close the bottom (i.e., distal) end following a portion of the vitrification preparation, in order to contain fluid within the straw to surround the population of oocytes, embryos or blastocysts with said fluid. In some embodiments, a filter is placed at a location inside the straw allowing flow of vitrification fluid to pass while retaining any desired material within the tube.

In some embodiments, the straw is about 1.0 inches to about 5.0 inches long. In some embodiments, the straw is made of polycarbonate. In some embodiments, the filter is affixed in the straw and comprises a plurality of pores having a diameter smaller than a diameter of said embryos or blastocysts but large enough to allow the passage of a fluid composition. In some embodiments, the plurality of pores in the filter have diameters from about 0.0001 inches to about 0.002 inches. It is to be understood that any means that prevent sliding of the filter inside the tube can be employed in the device of the present invention. In such a situation, the diameter gradient between the center portion and the proximal and distal ends may not be warranted. For example, the filter can be affixed in the tube with a tube extrusion that may tighten the fixation of the filter in the tube thereby preventing its sliding.

The fluid composition can include, without limitation, a cryogenic liquid, a cryoprotectant, a pretreatment medium, a dehydrant, a reanimating solution or combinations thereof. In some embodiments, the fluid compositions include solutions which do not form ice in liquid nitrogen. Suitable cryoprotectants or pretreatment mediums for use in vitrification are well known in the art and include, by way of example only, water, saline solutions such as phosphate-buffered saline, dimethyl sulfoxide (DMSO), ethylene glycol, propylene glycol (1,2-propanediol), glycerol, as well as mixtures of two or more of such, and the like. Cryogenic liquids include both liquids and gases capable of providing cryogenic temperatures for vitrification. Examples of cryogenic liquid include, without limitation, liquid argon, liquid nitrogen, liquid oxygen, liquid hydrogen or any other cryogenic liquid with suitable properties, and combinations thereof. Suitable dehydrating agents are known in the art and include, without limitation, solutions of sucrose, dextrose, trehalose, lactose, raffinose, and combinations thereof. In some embodiments, the fluid composition is a cryogenic liquid, a pretreatment medium or a dehydrant. In some embodiments, the cryogenic liquid is liquid nitrogen. In some embodiments, the fluid compositions include one or more of the fluids disclosed in International Publication No. WO2010141317, entitled “Populations of substantially spherical, reduced volume oocytes”, said publication herein incorporated by reference in its entirety.

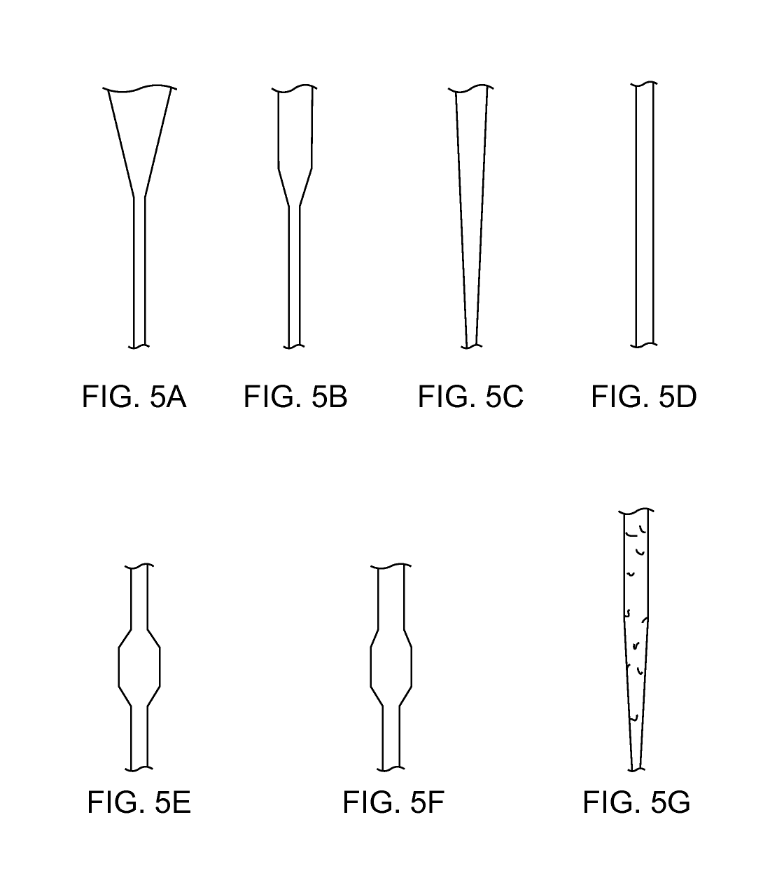

In some embodiments, the straw may further include one or more screens or sieves 610, as shown, for example, in the straw 600 of FIG. 6. It will be appreciated by those skilled in the art that while one straw design is shown in FIG. 6, the straw design was selected for illustrative purposes only, and the described screens or sieves 610 may be located within any straw embodiment described elsewhere herein or any other straw design contemplated by this invention. In some embodiments, the one or more sieves 610 are disposed proximal to the filter 605. In some embodiments, the straw of the device comprises a deposit chamber where the oocytes, embryos or blastocysts are deposited. For example, the filter assembly of some embodiments, such as the filter assembly 104 of FIG. 1B may act as a deposit chamber.

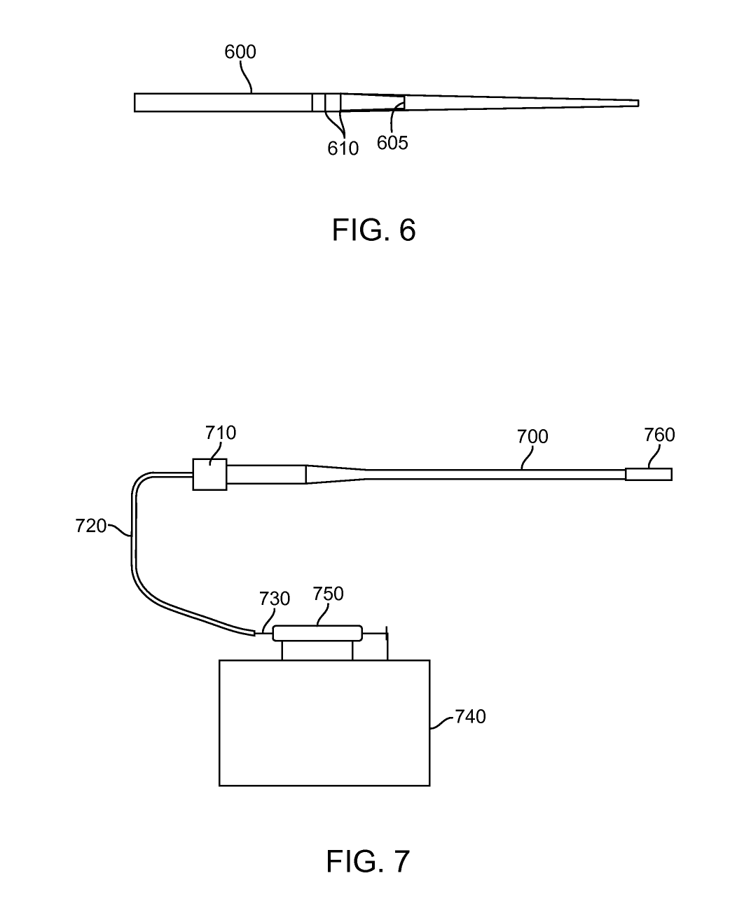

In some embodiments, connectors of various design may be provided and used to connect one or both ends of the straw to other devices. For example, in some embodiments, the device is affixed directly or indirectly to a source of a fluid composition. In some embodiments, the source is a syringe. The syringe of some embodiments may be inserted directly into the proximal end of the straw. In other embodiments, such as the embodiment of FIG. 7, a connector 710 is used to connect the straw of the vitrification device 700 to tubing 720, said tubing 720 attached to a syringe 730 engaged on a syringe pump 740. In some embodiments, the syringe 730 includes a container 750 (e.g., a syringe body), which holds a fluid composition, and the syringe 730 is capable of providing a steady or a pulsatile flow of the fluid composition through the device. In some embodiments, the flow through the syringe is powered by a stepper motor or a pump. It is to be understood that any means for generating a steady or a pulsatile flow of the fluid composition through the straw may be used. In some embodiments, the straw 700 may also connect directly or indirectly to a syringe, a pipette or other transfer device to gently deliver oocytes, embryos or blastocysts into the lumen of the straw 700. It is also to be understood that the schematic of FIG. 7 is not drawn to scale.

### 4. METHODS

Disclosed herein are methods for vitrifying and/or reanimating oocytes, embryos or blastocysts. The method of some embodiments includes: (a) placing one or more of oocytes, embryos or blastocysts on a filter affixed inside a straw, and (b) continuously passing a fluid composition through the straw and over the oocytes, embryos or blastocysts. The method can be initiated by placing the oocytes, embryos or blastocysts in the device, for example, by gently inserting them via a pipette, syringe, or other transfer device. Suitable fluid composition is then allowed to flow, through the straw. This fluid composition can be passed using any means that can pass the composition through the device, such as, but not limited to, a syringe, dropper, etc. In some embodiments, the fluid composition is allowed to flow through the device in a steady flow. In other embodiments, the fluid composition is allowed to flow through the device in pulses. In one embodiment, the solution flows from the proximal end to the distal end of the straw. In some embodiments, after one or more pre-vitrification solutions flow through the straw, an optional cap or other occlusion device is placed so as to occlude the distal end of the straw. Placement of the occlusion device allows for fluid to remain within the straw such that the oocytes, embryos or blastocysts may be fully covered and bathed in the fluid.

In one embodiment of the method, the straw has a lumen traversing through the straw and has a proximal section, a middle section and a distal section, which middle section is tapered from the proximal section to the distal section so that the proximal portion of the middle section has the same diameter as the proximal section and the distal portion of the middle section has the same diameter as the distal section, and which distal section is optionally capped to close the lumen running through the said straw. The filter comprises a plurality of pores having a diameter smaller than a diameter of said embryos or blastocysts, but large enough to allow the passage of the fluid composition.

The fluid flow can be adapted to be in any desired direction, i.e., from the proximal end to the distal end or from the distal end to the proximal end of the straw. In one embodiment, the fluid composition flows from the proximal end to the distal end of the straw. The fluid composition is optionally modified over time in a continuous manner so that the final fluid composition corresponds to that required for cryopreserving and/or reanimating of oocytes, embryos or blastocysts. Exemplary fluid compositions are provided in International Publication No. 102010141317, entitled “Populations of substantially spherical, reduced volume oocytes”, said publication herein incorporated by reference in its entirety. In one embodiment of the method, the flow of the fluid composition is adapted to allow continuous change in the osmolarity of the fluid composition in contact with the oocytes, embryos or blastocysts.

In certain embodiments, the oocytes, embryos or blastocysts are contacted with the cryoprotecting or reanimating solutions described herein for a predetermined period of time to obtain the desired portion of a cryoprotectant or water in the oocytes, embryos or blastocysts thereby producing a population of substantially spherical oocytes, embryos or blastocysts. Further, the rate of flow and change in osmolarity of the fluid composition is maintained under conditions to retain sphericity of the oocytes, embryos or blastocysts.

The ratio of the volume of the fluid composition to the number of oocytes, embryos or blastocysts is adapted to obtain the desired osmolarity with the passage of a minimum amount of the fluid composition. In some embodiments, the ratio of the volume of the fluid composition to the number of oocytes, embryos or blastocysts ranges from about 2:1 to about 100:1.

The devices and methods disclosed herein are useful for maintaining a population of substantially spherical oocytes, embryos or blastocysts during vitrification or reanimation. In some embodiments, the volume ratio of the fluid composition to the oocyte, embryo and/or blastocyst is sufficient to allow vitrification of the oocyte, embryo and/or blastocyst with substantial retention of sphericity. In various embodiments, the ratio of the volume of the fluid composition to number of oocytes, embryos or blastocysts is sufficient to maintain about 99%, about 95%, about 90%, about 80%, about 85%, about 80%, about 75%, about 70%, about 65%, about 60%, or about 55% sphericity of the oocytes, embryos or blastocysts. In some embodiments, the ratio of the volume of the fluid composition to number of oocytes, embryos or blastocysts is sufficient to maintain 70% sphericity of the oocytes, embryos or blastocysts. The fluid composition can be a cryogenic liquid, a cryoprotectant, a pretreatment medium, or a dehydrant, a reanimating solution, or combinations thereof as described herein. In some embodiments, the cryogenic liquid is liquid nitrogen.

In some embodiments, upon capping the straw filled with a cryoprotectant, a pretreatment medium, and/or other solution, the straw is inserted into a protective sleeve and the sleeve is quickly submerged into a cryogenic liquid, such as, for example, liquid nitrogen. In some such embodiments, the liquid nitrogen travels up through the lumen of the protective sleeve, surrounding the straw, and resulting in rapid cooling of the straw's contents, including the oocytes, embryos, or blastocysts disposed therein. In some embodiments, the cooling of the contents to a glass state is nearly instantaneous, for example, less than 10 seconds, less than 5 seconds, less than 2 seconds, less than 1 second, less than 0.5 seconds, less than 0.1 seconds, or less than 0.01 seconds. Through such embodiments, vitrification of oocytes, embryos and blastocysts can be achieved while maintaining the degree of sphericity needed to maintain viability.

In some embodiments, the method further comprises providing data from one or more sensors responsive to one or more parameters related to the vitrification and/or reanimation method. For example, the sensors can be configured to measure sphericity of the oocytes, embryos or blastocysts throughout the vitrification or reanimation process. Sphericity is a measure of the roundness of the oocyte, embryo or blastocyst and can be defined by the ratio of the surface area of a sphere, having a volume equal to the oocyte, embryo or blastocyst volume, to the surface area of the oocyte, embryo or blastocyst. Sphericity can also be approximated by circularity of the oocyte, embryo or blastocyst. Volume and surface area of the oocyte, embryo or blastocyst can be determined from measurement systems known in the art such as ultrasound and optical imaging systems.

### 5. KIT OF PARTS

In one aspect of the invention, there is provided a kit comprising any embodiment of the device described herein and an apparatus that flows the fluid composition into the device. The apparatus that flows the fluid composition can be a reservoir such as a bag, a glass vial, a container, or a cartridge, a syringe or a dropper. The kit may further comprise a motor that can be attached to the apparatus or can be provided separately to be attached to the reservoir, syringe or dropper at the time of operation. The kit may additionally or alternatively comprise a container, such as a bottle, an ampule or a syringe, containing a fluid composition such as a cryoprotecting solution, a reanimating solution or both. The kit may additionally or alternatively comprise an instruction sheet for using the parts. In some embodiments, the kit comprises usual operational tools such as forceps, gloves, petri dishes, etc.

As shown in the schematic diagram of FIG. 7, in some embodiments, the kit includes a vitrification device 700 formed of a straw and filter, such as, for example, any embodiment of the vitrification device disclosed elsewhere herein or any other embodiment contemplated by this invention. The device 700 may be boxed or otherwise packaged with a protective sleeve, for example, protective sleeve 120 of FIG. 1F or protective sleeve 220 of FIG. 2D. The vitrification device may also be packaged with a user manual and/or instructions for use. The kit may also include a removable stopper or cap 760. The kit of some embodiments further includes one or more of the fluid compositions used with the vitrification device 700 to implement the vitrification process; the kit may include one or more cryoprotecting solutions or reanimating solutions packaged within a bottle, vial, or other container 750, such as, for example, the container 750 that forms a syringe body. In some embodiments, the kit also includes a syringe 730 and/or tubing 720 or other apparatus for delivering the fluid composition(s) and/or the sample of oocytes, embryos or blastocysts into the vitrification device. Additionally or alternatively, the kit may include a connector device 710, which attaches to the vitrification device 700 via, for example, a friction fit, snap fit, or threaded engagement, and which connects the vitrification device 700 to other devices. In some embodiments, the kit may also include a syringe pump 740 or other motorized unit for pumping fluid from the container, syringe, or dropper into the vitrification device 700.

It is to be understood that while the invention has been described in conjunction with the above embodiments, that the foregoing description and examples are intended to illustrate and not limit the scope of the invention. Other aspects, advantages, omissions, substitutions and modifications may be made without departing from the scope of the invention. Therefore, the scope of the invention is defined by the claims that follow rather than by the foregoing description. All variations coming within the meaning and range of equivalency of the claims are embraced within their scope.

What is claimed is:

1. A device adapted for vitrification of an oocyte, embryo or blastocyst having a defined diameter, which device comprises:
a straw; and
a filter;
wherein the straw comprises a lumen traversing through the straw and has a proximal section, a middle section and a distal section; which middle section is tapered from the proximal section to the distal section so that the proximal portion of the middle section has the same diameter as the proximal section and the distal portion of the middle section has the same diameter as the distal section; and which distal section is optionally capped to close the lumen running through said straw;
wherein the filter is affixed in the straw and comprises a plurality of pores having a diameter smaller than the diameter of said oocyte, embryo or blastocyst but large enough to allow the passage of a fluid composition therethrough;
wherein the straw, when capped, has an interior volume that allows the fluid composition to bathe the oocyte, embryo or blastocyst, wherein the volume ratio of the fluid composition to the oocyte, embryo or blastocyst is sufficient to allow vitrification of the oocyte, embryo or blastocyst with substantial retention of sphericity; and
further wherein at least a portion of the straw proximate to the filter is composed of non-insulating materials.

2. The device of claim 1, wherein the volume ratio of the fluid composition to the oocyte, embryo and/or blastocyst is from about 2:1 to 100:1.

3. The device of claim 1, wherein the filter is affixed in the distal section of the straw.

4. The device of claim 1, wherein the fluid composition is retained in the distal section when the distal end of the straw is capped.

5. The device of claim 1, wherein the fluid composition is retained in the distal and the middle section when the distal end of the straw is capped.

6. The device of claim 1, wherein the straw is about 1.0 inches to about 5.0 inches long.

7. The device of claim 1, wherein the straw is made of polycarbonate or polyethylene terephthalate.

8. The device of claim 1, wherein the plurality of pores in the filter have diameters from about 0.0001 inches to about 0.002 inches.

9. The device of claim 1, wherein the filter holds a plurality of oocytes, embryos or blastocysts.

10. The device of claim 1, wherein the filter is made of polycarbonate or nylon.

11. The device of claim 1, wherein the fluid composition is a cryogenic liquid, a pretreatment medium or a dehydrant.

12. The device of claim 11, wherein the cryogenic liquid is liquid nitrogen.

13. A device adapted for vitrification of oocytes, embryos or blastocysts, which device comprises:
a straw; and
a filter;
wherein the straw has a lumen traversing through the straw and comprises at least two sections: a proximal section and a distal section; wherein said straw is tapered through the distal section; wherein the proximal section has an internal diameter of 0.05 inches to 0.07 inches and an external diameter of 0.05 inches to 0.09 inches, and wherein at least a portion of the distal section has an internal diameter of 0.01 inches to 0.04 inches and an external diameter of 0.015 inches to 0.04 inches, and the portion between the external diameter and the internal diameter comprises a wall of the straw; and
wherein the filter is affixed in the straw and comprises a plurality of pores having a diameter smaller than a diameter of said oocytes, embryos or blastocysts but large enough to allow the passage of a fluid composition.

14. The device of claim 13, wherein the wall of the straw has a thickness of about 0.002 inches to about 0.02 inches.

15. The device of claim 13, wherein the wall of the straw is adapted to be thermally conductive and mechanically resistant to pressure.

16. A method for vitrification of oocytes, embryos or blastocysts, comprising:
(a) placing one or more of oocytes, embryos or blastocysts on a filter affixed inside a straw, and
(b) continuously passing a fluid composition through the straw and over the oocytes, embryos or blastocysts;
wherein the straw comprises a lumen traversing through the straw and has a proximal section, a middle section and a distal section; which middle section is tapered from the proximal section to the distal section so that the proximal portion of the middle section has the same diameter as the proximal section and the distal portion of the middle section has the same diameter as the distal section; and which distal section is optionally capped to close the lumen running through said straw;
wherein the filter comprises a plurality of pores having a diameter smaller than a diameter of said oocytes, embryos or blastocysts but large enough to allow the passage of the fluid composition;
wherein a ratio of the volume of the fluid composition to number of oocytes, embryos or blastocysts is adapted to obtain a desired osmolarity with the passage of a minimum amount of the fluid composition; and
wherein the fluid composition is optionally modified over time in a continuous manner so that the final fluid composition corresponds to that required for vitrification of oocytes, embryos or blastocysts.

17. The method of claim 16, wherein the fluid composition flows from the proximal end to the distal end of the straw.

18. The method of claim 16, wherein flow of the fluid composition is adapted to allow continuous change in the osmolarity of the fluid composition in contact with the oocytes, embryos or blastocysts.

19. The method of claim 18, wherein a rate of flow and change in osmolarity of the fluid composition is maintained under conditions to retain sphericity of the oocytes, embryos or blastocysts.

20. The method of claim 16, wherein the ratio of the volume of the fluid composition to number of oocytes, embryos or blastocysts ranges from about 2:1 to about 100:1.

21. The method of claim 16, wherein the ratio of the volume of the fluid composition to number of oocytes, embryos or blastocysts is sufficient to maintain 70% sphericity of the oocytes, embryos or blastocysts.

22. The method of claim 16, wherein the fluid composition is a cryogenic liquid, a pretreatment medium or a dehydrant.

23. The method of claim 22, wherein the cryogenic liquid is liquid nitrogen.

24. The method of claim 16, further comprising providing data from one or more sensors responsive to one or more parameters related to the vitrification and/or reanimation method.
